(19) United States
(12) Patent Application Publication
(10) Pub. No.: US 2025/0130223 A1
(43) Pub. Date: Apr. 24, 2025

(54) IN VITRO OR EX VIVO METHOD FOR DETERMINING THE EFFECT OF A BIOLOGICAL SAMPLE ON A BIOLOGICAL MODEL USING LARGE-SCALE NEURAL ACTIVITY

(71) Applicant: NETRI, Lyon (FR)

(72) Inventors: Louise MINY, LYON (FR); Lucas ARBABYAZD, VILLEURBANNE (FR); Florian Arnaud Paul LARRAMENDY, DARDILLY (FR); Thibault HONEGGER, LYON (FR)

(21) Appl. No.: 18/836,873

(22) PCT Filed: Feb. 9, 2023
(86) PCT No.: PCT/FR2023/050180

§ 371 Date: Aug. 8, 2024

(30) Foreign Application Priority Data
Feb. 9, 2022 (FR) FR22/01131

(51) Int. Cl. G01N 33/50; C12M 1/00; C12M 1/34; G01N 21/64

(52) U.S. Cl. CPC G01N 33/5058; C12M 23/58; C12M 41/46; G01N 21/6456

(57) ABSTRACT

An in vitro or an ex vivo method for determining the effect of a biological sample on a biological interface in particular, includes the implementation of a bioreceptor having a multi-compartment microfluidic device incorporating a relevant cell co-culture to which the sample is applied. The response of the neural network to the sample, in particular a change in the cell/neural network, is recorded and subsequently analyzed. A differential diagnosis is subsequently carried out by comparing the network markers for true positives and the test samples.

### CROSS-REFERENCE TO RELATED APPLICATIONS

This application is a 35 U.S.C. §371 National Stage patent application of PCT/FR2023/050180, filed on 9 Feb. 2023, which claims the benefit of French patent application 22/01131, filed on 9 Feb. 2022, the disclosures of which are incorporated herein by reference in their entirety.

### TECHNICAL FIELD

The disclosure relates to the field of diagnosis, in particular that of diagnosis bioreceptors, associated with a microfluidic technology as biosensors. Thus, the disclosure relates to a method for determining the effect of a biological sample on a biological model, to a bioreceptor implemented in such a method as well as to the uses of said bioreceptor.

### BACKGROUND

The pathologies of the central and/or peripheral nervous system affect more than 700 million people worldwide, more than 10 million in Europe and tens of thousands in France. These pathologies are considered to be the most complex in terms of etiology, evolution but also treatment. These neurocognitive disorders (for example, the Alzheimer's disease, the Parkinson's disease, cranial traumas, cerebral vascular accidents, or amyotrophic lateral sclerosis), lead to a cognitive, functional and/or behavioral deficit of a subject, that is to say an alteration of the capabilities related to language, to social interactions, to memory, to logical reasoning or to autonomy. Hence, these pathologies, also so-called neurological or nerve conditions, constitute a major public health problem.

Currently, the diagnosis of these conditions is done essentially after the apparition and the observation of the first symptoms. Following this first clinical diagnosis, assays of biological fluids, such as blood or cerebrospinal fluid (CSF), supplemented by imaging tests are carried out. As regards other neuronal disorders, such as cranial traumas (or concussions), the diagnosis is based on the establishment of a Glasgow score established by clinical observations and symptoms.

In recent years, the presence of novel biomarkers allowing diagnosing these neurological or nerve conditions has been demonstrated. For example, microRNAs present in biological fluids such as LCS or blood allow diagnosing concussions via very conventional techniques of biological assays like polymerase chain amplification (or polymerase chain reaction or PCR) or the immunoenzymatic technique on a solid support (or enzyme-linked immunosorbent assay or ELISA).

The current treatments are carried out in the context of a secondary and tertiary accompanying and prevention approach intended to preserve the life quality, prevent complications and behavioral seizures by anticipating the advanced stages of the diseases. In addition, the search for new treatment is made difficult by the complexity of establishment of a reliable differential diagnosis. Indeed, for several different impairments such as neurodegenerative diseases, there might be identical or similar clinical symptoms due to the involvement of the same dysfunctional proteins in these disorders.

Hence, in order to ensure efficient and earliest management of the disease, it is essential to establish a reliable and early diagnosis of such a neurological or nerve condition and thus be able to improve the identification of a suitable treatment.

However, to date, there is no sensitive, rapid and reproducible test enabling the early and deterministic diagnosis of a cognitive, functional and/or behavioral deficit associated with a neurological and/or nerve condition.

In parallel, methods for assaying the activity of an agent, for example a drug, on the neurological activity are generally based on behavioral alterations of living animals or use tissue biosensors. However, this type of behavioral tests on animal models turns to be costly, long, difficult to quantify and barely reproducible.

The use of tissue-based biosensors overcomes some of the limits imposed by behavioral tests and provide a result whose quantization is easier. However, such biosensors have limited sensitivity and cannot detect subtle alterations of the cognitive function. Tissue biosensors capable of detecting agents that alter or otherwise modify the neural function generally consist of cultured neurons held on a network of electrodes that record the passive properties of the cellular membrane, like the input impedance, or the activity of the spontaneous action potential. Because of a low sensitivity, these types of biosensors are essentially implemented for the determination of acute cell death due to exposure to high concentrations of toxic agents (for example, excitotoxicity induced by high concentrations of glutamate in the synaptic slot). In addition, most biosensors provide only short-term data.

In general, the conventional solutions have the drawbacks/) they do not allow, or barely allow, the detection of the presence of unsuspected or new agents which could be at the origin or indicate the presence of a neurological and/or nerve condition;/) they essentially detect the effect of rapid acting agents while the agents that require several hours or days to produce their effect cannot generally be detected with known methods; and///) they do not enable a distinction of the affected neural populations.

It arises from the foregoing that there is an obvious need to develop new solutions enabling a rapid, efficient, early and reliable differential diagnosis of neurological and/or nerve conditions.

### BRIEF DESCRIPTION OF THE DISCLOSURE

The Inventors have unexpectedly and surprisingly developed a method for determining the effect of a biological sample on a biological model involving a neural network. This method is implemented by means of an innovative biosensor comprising in particular a bioreceptor comprising, advantageously in the form of, a multi-compartment microfluidic device. This method, including the bioreceptor for determining the effect of a biological sample on a biological model and its uses, are characterized throughout the present description.

An object of the present disclosure is to obtain data on the state of the neural network exposed to a biological sample and then to analyze these data in order to establish, rapidly and at lower cost, a differential diagnosis of a neurological and/or nerve condition in a subject with an improved specificity/sensitivity ratio. In fine, the aim is to provide a treatment suited to the subject aiming at preserving the life quality, preventing complications and anticipating the evolution of the pathology, in particular towards advanced stages of the pathologies.

Thus, the present disclosure relates to an in vitro or ex vitro method for determining the effect of a biological sample on a biological model, comprising the following steps of:

a. Providing a bioreceptor comprising, advantageously in the form of, a multi-compartment microfluidic device comprising:

i) at least a first compartment and a second compartment;

ii) at least one means forming a biological interface for enabling communication by neural connection between the first and second compartments;

iii) the culture of at least one type of cells or explant per compartment, the first compartment comprising at least the neuron culture in the form of a neural network and the second compartment comprising at least the neuron culture in the form of a neural network and/or the culture of non-neuronal cells or an explant culture;

iv) at least one device enabling recording of the functional activity of the neurons over a plurality of measurement points spatially distributed in the first compartment;

b. Directly or indirectly contacting the neurons and/or non-neuronal cells or the explant in culture in the second compartment with said biological sample;

c. Carrying out a recording of the functional activity of the neurons in culture in the first compartment over the plurality of measurement points over a measurement duration following contacting according to step b);

d. Carrying out a conversion of the record of the functional activity of the neurons in culture in the first compartment into functional activity data;

e. Analyzing the functional activity data obtained in step d);

f. Determining at least one characteristic parameter of the state of the neural network in the first compartment from the functional activity data;

g. Carrying out a comparison between the at least one characteristic parameter of the state of the neural network and a reference value of at least one characteristic parameter of the state of the neural network in order to determine the effect of said biological sample.

In the remainder of the disclosure and for simplicity:
the term “neuron” is used equivalently with “neural cells”;
the term “neurological and/or nerve impairment” is used equivalently with “pathology of the central and/or peripheral nervous system” or “neurocognitive impairment” or “cognitive, functional and/or behavioral deficit”; and
the term “impairment” is used equivalently with “pathology”, “attainment” or “lesion”.

In the context of the present disclosure, by “biological model”, it should be understood the culture of non-human, human cells derived from primary pluripotent or human stem cells, alone or in co-culture, neuronal or non-neuronal structures isolated from their natural biological environment, in a multi-compartment microfluidic architecture wherein the compartments are connected directly or indirectly. In other words, this consists in culturing cells in an artificial environment with a minimum of alteration of the natural conditions or in vivo conditions.

In the context of the present disclosure, by “biological interface”, it should be understood a system comprising a contact junction between cell populations included in the device of the disclosure enabling communication of the cells by exchanges of information via an electrical and/or chemical communication. Within the meaning of the disclosure, this may consist of a culture of neuronal cells, non-neuronal cells or a tissue explant.

In the context of the present disclosure, by “bioreceptor”, it should be understood a set of molecules and/or cells enabling the selective recognition of a molecule (or analyte). For example, mention may be made of enzymes, cells, aptamers, nanoparticles or antibodies. Within the meaning of the disclosure, the bioreceptor is a culture of neurons, for example human neurons derived from pluripotent stem cells, included within a multi-compartment microfluidic device modeling a network architecture which could be characteristic of a neurological and/or nerve pathology. This bioreceptor is associated with a transducer which will convert the association of the analyte and the bioreceptor into a measurable signal. According to the disclosure, this combination of a bioreceptor and a transducer is defined as a biosensor whose advantage is the establishment of a rapid and reliable diagnosis, in particular thanks to the presence of a biological interface within these microfluidic architectures which makes the bioreceptor more physiological, that is to say that it consists of an innovative experimental model whose physical, biochemical and biological organization, operation and reactions allow transposing the obtained results to humans. In the context of the present disclosure, by “biosensor”, it should therefore be understood a device comprising at least one bioreceptor as defined before and a means for converting the functional activity of the neurons into functional activity data as defined later on.

In the context of the present disclosure, by “functional activity of the neurons”, it should be understood the emission and propagation of a nerve message in the form of electrical signals and/or neurotransmitter secretions.

In the context of the present disclosure, by “a means for converting the functional activity of the neurons into functional activity data”, it should be understood a transducer. Transducers may be grouped into 3 large categories: Optical (for example, optical fiber), electrochemical (for example amperometric, potentiometric, impedimetric or conductimetric), and based on mass (for example piezoelectric and magnetoelastic).

In the context of the present disclosure, by “node”, “node link” or “vertices/edge”, it should be understood a neuron, an assembly of cells or of population of neurons linked in a continuous manner and having a functional activity. It should be noted that the brain comprises about 100 billion neurons and 1 neuron can establish up to 10,000 connections.

In the context of the present disclosure, by “module”, it should be understood a plurality of nodes connected together forming groups or “clusters”.

In the context of the present disclosure, by “action potentials” or “spikes”, it should be understood the apparition of nerve influx, namely the succession of a transient and local depolarization of the plasma membrane and then a repolarization of the inner membrane (possibly followed by a hyperpolarization for non-myelinated cells).

In the context of the present disclosure, by “burst(s)”, it should be understood several action potentials within a given time period. A burst is generally defined by a succession of at least four action potentials within a defined time period, for example, for 100 ms. A burst generally contains between 10 and 100,000 spikes for such a duration.

In the context of the present disclosure, by “network burst(s)”, it should be understood groups of action potentials forming a network, that is to say according to a defined apparition synchrony. In other words, the network bursts are detected by the synchronous action potentials of several electrodes.

Preferably, the present disclosure relates to an in vitro or ex vivo method for determining the effect of a biological sample on a biological model as defined before having the following technical features, considered separately or in combination:
the at least one characteristic parameter of the state of the neural network in the first compartment being selected from the group consisting of: a number of action potential(s), an action inter-potential interval or “ISI”, a variation coefficient of inter-action potential interval(s), a number of active electrodes, an average rate of normalized action potentials, a number of bursts, a number of electrode(s) having captured one or several burst(s), an average duration of the bursts, an average of action potentials (ISI) in a burst, an average of the inter-action potential intervals in a burst, an inter-burst interval or “IBI”, a frequency of the bursts, a percentage of the bursts, a number of network bursts, a network burst frequency, a network burst duration, an average of action potentials in network bursts, an average of the inter-action potential intervals (ISI) in network bursts, a number of electrodes participating in forming network bursts, a burst percentage in network bursts, an inter-burst interval (IBI) variation coefficient in network bursts, a surface area under the cross-correlation curve, and a synchrony index,
the at least one characteristic parameter of the state of the neural network in the first compartment being selected from the group consisting of:

i) a connection coefficient;

ii) an average of the inter-node minimum lengths;

iii) an average of the action potentials per second;

iv) a network connectivity index or “Small World Index”;

v) a z score or “z-score”;

vi) a participation coefficient or “Participation Coefficient”; and

vii) a centrality index of a node,
the biological interface comprises, advantageously consists of at least one of the elements selected from the group consisting of fluidic microchannels; PDMS microchannels; a porous membrane, whose porosity is advantageously comprised between 10 nm and 40 μm and whose pore density is advantageously comprised between 10 and 1.10⁹ pores per cm², advantageously between 1.10⁵ and 1.10⁹ pores per cm²; a porous capillary membrane, advantageously it consists of a membrane on which at least one organoid is cultured (i.e. a three-dimensional multicellular structure which reproduces in vitro the microanatomy of an organ) made of polycarbonate, polyester, polyethylene terephthalate and/or polytetrafluoroethylene; a gel; a hydrogel and mixtures thereof;
the construction of a graph representing the neural network in step e), which is obtained by implementing the graph theory; advantageously the nodes of the graph correspond to the measurement points of the functional activity and the connections between nodes correspond to the correlations of the axonal communications;
step f) comprises determining two parameters, advantageously three parameters, four parameters, preferably five parameters, six parameters, and even seven characteristic parameters of the state of the neural network in the first compartment from the functional activity data, said parameters being selected from the group consisting of:

i) a connection coefficient;

ii) an average of the inter-node minimum lengths;

iii) an average of the action potentials per second;

iv) a network connectivity index or “Small World Index”;

v) a z score or “z-score”;

vi) a participation coefficient or “Participation Coefficient”; and

vii) a centrality index of a node;
the connectivity index of the network is the ratio between the connection coefficient and the average of the inter-node minimum lengths;
the average of the action potentials per second should have a value greater than or equal, advantageously strictly greater, than 0.5, preferably 1 or 1.5, and even 2, to enable the analysis of at least one parameter selected from the group consisting of:

i) a connection coefficient;

ii) an average of the inter-node minimum lengths;

iv) a network connectivity index or “Small World Index”;

v) a z score or “z-score”;

vi) a participation coefficient or “Participation Coefficient”; and

vii) a centrality index of a node;
the connection coefficient has a value greater than or equal to 0, preferably 0.5, advantageously comprised between 0 and 1;
the average of the inter-node minimum lengths has a value greater than or equal to 1, preferably 1.5 or 2;
the connectivity index of the inter-node network has a value greater than or equal to 0, preferably 0.5, advantageously comprised between 0 and 1;
the score z or “z-score” has a value greater than or equal to 0, preferably 5, advantageously comprised between 0 and 10;
the participation coefficient or “Participation Coefficient” has a value greater than or equal to 0, preferably 0.5, advantageously comprised between 0.5 and 1;
the centrality index of a node has a value greater than or equal to 0, preferably 5, advantageously comprised between 6 and 10;
the device allowing recording the functional activity of the neurons over a plurality of measurement points spatially distributed in the first compartment according to step a.iv), preferably spatially distributed in the first compartment and at least one additional compartment, advantageously spatially distributed in all compartments, is a device enabling an indirect contact recording with the cultured cells, selected from the group consisting of:
a device for recording activity by arrays of planar or non-planar microelectrode, semi-solid electrodes, by amperometry or voltammetry;
a fluorescence imaging recording device, such as calcium imaging or transmembrane ion flow imaging; and
a device for recording intracellular, extracellular, or patch-clamp electrophysiological activity in whole cell, attached cell, inside-out or outside-out configuration;
step d) of carrying out a conversion of the record of the functional activity of the neurons in culture in the first compartment into functional activity data is carried out via a means for converting the functional activity of neurons into functional activity data is an algorithmic system for converting electrical and/or electrophysiological data into binary data;
the duration of measurement of the record of the functional activity of the neurons in culture in the first compartment according to step c) is comprised between 300 ms and 20 min, advantageously between 1 min and 15 min, preferably between 5 min and 12 min;
the multi-compartment microfluidic device further comprises a third compartment and at least one means forming a biological interface to enable communication by neural connection between the first and third compartments and/or at least one means forming a biological interface to enable communication by neural connection between the second and third compartments; advantageously, a fourth compartment and at least one means forming a biological interface to enable communication by neural connection between the first and fourth compartments and/or at least one means forming a biological interface to enable communication by neural connection between the second and fourth compartments and/or at least one means forming a biological interface to enable communication by neural connection between the third and fourth compartments; preferably, a fifth compartment and at least one means forming a biological interface to enable communication by neural connection between the first and fifth compartments and/or at least one means forming a biological interface to enable communication by neural connection between the second and fifth compartments and/or at least one means forming a biological interface to enable communication by neural connection between the third and fifth compartments and/or at least one means forming a biological interface to enable communication by neural connection between the fourth and fifth compartments;
the contacting according to step b) is indirect in that the biological sample is applied on the biological interface of the second compartment and/or on at least one of the biological interfaces of the third compartment and/or on at least one of the biological interfaces of the fourth compartment and/or on at least one of the biological interfaces of the fifth compartment;
each of the compartments included in the device of the disclosure comprises the culture of one, two or even three types of neuronal and/or non-neuronal cells;
the neurons are selected from the group consisting of glutamatergic, GABAergic, serotoninergic, cholinergic, dopaminergic, adrenergic, noradrenergic, sensory neurons and motor neurons;
the non-neuronal cells are selected from the group consisting of glial cells (comprising microglia/macrophages and macroglia (i.e. oligodendrocytes, Schwann cells, and ependymocytes)), epithelial, conjunctive, thyroid, fat, blood, immune, bone, cartilaginous, gastric, pancreatic, hepatic, intestinal or pulmonary cells, endothelial, muscular, vascular, cardiac, mesenchymal cells, cells of the retinal pigment epithelium and retinal cells;
the explant is a tissue of cerebral, epithelial, ocular, thyroid, fatty, vascular, bone, cartilaginous, gastric, pancreatic, hepatic, intestinal, pulmonary, endothelial, muscle and retinal origin, cardiac and placental; and/or
the biological sample is selected from the group consisting of blood, saliva, urine, tears, sweat, sputum, mucus, pus, lymph, cerebrospinal fluid, nasopharyngeal secretions, oropharyngeal secretions, synovia, pleural fluid, peritoneal fluid, pericardial fluid, aqueous humor, amniotic fluid and plasma; and/or - the biological sample may be an “agent” or a “test agent”, that is to say a compound having modulatory properties of the functional activity of the neurons or an agent that we do not know whether or not it has modulatory properties of the functional activity of the neurons, in which case the method of the disclosure allows identifying and/or characterizing possible properties of said agent, and even identifying a “threshold concentration”. In other words, in particular when the test agent is a medicine, this consists of the concentration of the agent under a minimum treatment regime (i.e. for a pharmacological composition, under the therapeutic dosage generally prescribed the lowest for animals or humans). In this embodiment of the disclosure, the comparison with a reference value carried out in step g) consists in comparing at least one characteristic parameter of the state of the neural network of the disclosure consisting of a value obtained before application of the agent or test agent and/or with a value obtained after application of the agent or test agent, said value being earlier in time to ensure monitoring of the effect of said agent or test agent.

Thus, in the context of the present disclosure, by:
“number of action potential(s)”, it should be understood the number of action potentials detected during recording (detection parameters defined upstream of recording);
“inter-action potential interval” or “ISI”, it should be understood the average duration between the detected action potentials;
“variation coefficient of inter-action potential interval(s)”, it should be understood the standard deviation of the ISIs divided by the average of the ISIs. This parameter relates to the regularity of the action potentials and reflects their distribution;
“number of active electrodes”, it should be understood the number of electrodes which have an activity greater than or equal to a minimum average rate of action potential, defined before recording,
“a normalized average rate of action potentials”, it should be understood the number of action potentials divided by the recording time (average). This parameter is generally defined in Hz or spike/s (action potential/second). This parameter is determined only from the functional activity data measured on the active electrodes, defined before recording;
“number of bursts”, it should be understood the number of bursts in a record per electrode or in total (sum of the bursts of all electrodes),
“number of electrodes that have captured bursts”, it should be understood the total number of electrodes that have measured a number of bursts/minutes. This number of bursts/minute may be defined before recording. For example, this may consist of at least 5 bursts/minutes;
“average duration of the bursts”, it should be understood the average time between the first and last action potentials defining each measured burst;
“average of action potentials in a burst” or “average of the spikes in a burst”, it should be understood the average number of the action potentials of the measured bursts;
“average of the inter-action potential intervals (ISI) in a burst”, it should be understood the average of the inter-action potential intervals defined in a burst;
“inter-burst interval” or “IBI”, it should be understood the average duration between the different recorded bursts;
“frequency of the bursts”, it should be understood the total number of bursts divided by the recording time. This parameter is generally defined in hertz or burst/min;
“percentage of the bursts”, it should be understood the ratio between the number of action potentials in a burst and the total number of recorded action potentials;
“number of network bursts”, it should be understood the total number of network bursts identified during recording;
“network burst frequency”, it should be understood the total number of network bursts divided by the recording time. This parameter is generally defined in hertz;
“network burst duration”, it should be understood the average time between two action potentials in a network burst;
“average of action potentials in burst networks”, it should be understood the average of the action potentials detected within all recorded burst networks;
“average of the inter-action potential intervals (ISI) in network bursts”, it should be understood the average of the inter-action potential intervals defined in all recorded network bursts;
“number of electrodes participating in forming network bursts”, it should be understood the average number of active electrodes in a network burst;
“burst percentage in network bursts”, it should be understood the ratio between the number of action potentials in a network burst and the total number of recorded action potentials;
“variation coefficient of inter-burst intervals (IBI) in network bursts”, it should be understood the standard deviation of the IBIs divided by the average of the IBIs;
“surface area under the cross-correlation curve”, it should be understood the surface area under the cross-correlation curve between the electrodes. This parameter may be determined according to the method defined by Halliday, Rosenber, Breeze & Conway 2006, in their publication “Neural Spike Train Synchronization Indices: Definitions, Interpretations, and Applications”;
“Synchrony index”, it should be understood a synchrony measurement (without unit) comprised between 0 and 1. This parameter may be determined in accordance with the publication “A comparison of binless spike train measures” (Paiva et al., 2010);
“connection coefficient” or “clustering coefficient”, it should be understood the probability that two nodes are connected knowing that they have a common neighbor;
“average of the inter-node minimum lengths”, it should be understood the average of the lengths between two connected nodes; and
“average of the action potentials per second”, it should be understood the average of the number of nerve influx per second, namely the succession of a transient and local depolarization of the plasma membrane and then a repolarization of the inner membrane (possibly followed by a hyperpolarization for the non-myelinated cells);
“connectivity index of the network” or “Small World index”, it should be understood a structural connection (physical connections, i.e. synapses and axons) and physiological connections (functional connectivity/symmetric relationship and effective connectivity/causal relationship) between two or more nodes, which is the reflection of the robustness of the neural network. Preferably, the connectivity index of the network is determined by the ratio between the connection coefficient and the average of the inter-node minimum lengths;
z score or “z-score”, it should be understood a measurement allowing characterizing the way in which the connectivity of the nodes is distributed in the modules, that is to say characterizing intra-modules;
participation coefficient or “Participation Coefficient”, it should be understood a measurement allowing characterizing the way in which the connectivity of the nodes is distributed between several modules i.e. inter-module; and
“centrality index of a node”, it should be understood a value proportional to the number of passages by this node during a random course over the graph, representing the neural network according to the disclosure, randomly following one of the connections starting from a node.

These characteristic parameters of the state of the neural network in the first compartment, determined from the recorded functional activity data, may be used alone or in combination. They are distributed into the following four categories:

1/ spikes (action potentials) analysis. This category comprises the following parameters: number of action potential(s), average of the action potentials per second, inter-action potential interval or “ISI”, coefficient of variation of inter-action potential interval(s), number of active electrodes, average rate of normalized action potentials;

2/ burst analysis. This category comprises the following parameters: number of bursts, number of electrode(s) having captured one or several burst(s), average duration of the average bursts of action potentials in a burst, average of the inter-action potential intervals (ISI) in a burst, inter-burst interval or “IBI”, frequency of the bursts and percentage of the bursts.

3/ Network Burst analysis. This category comprises the following parameters: network bursts number, network bursts frequency, network bursts duration, average of action potentials in network bursts, average of the inter-action potential intervals (ISI) in network bursts, number of electrodes participating in forming network bursts, percentage of bursts in network bursts, coefficient of variation of inter-burst intervals (IBI) in network bursts.

4/ network connectivity. This latter category applies to any type of networks, including neural networks. It comprises the following parameters, some of which are determined using connectivity algorithms so-called CrossCorrelation (cf. “Total spiking probability edges: A cross-correlation based method for effective connectivity estimation of cortical spiking neurons” Deblasi et al., 2019): surface area under the cross-correlation curve, synchrony index, connection coefficient, average of the inter-node minimum lengths, connectivity index of the network or “Small World Index”, participation coefficient or “Participation Coefficient”, and centrality index of a node.

In particular, the device allowing recording of the functional activity of the neurons over a plurality of measurement points spatially distributed in the first compartment according to step a.iv) comprises electrodes in direct or indirect contact with the neurons. This device allows recording the polarization difference of the neurons allowing communicating with one another by action potentials characterized by a potential difference higher than 1 μV.

The means for converting the functional activity into functional activity data according to step a.iv) of the disclosure is an algorithmic system for converting electrical and/or electrophysiological data into binary data. In particular, this consists in:

i) converting electrophysiological data by means of a signal transducer provided with the recording equipment, said transducer could be coupled with a signal amplifier;

ii) establishing a detection threshold to binarize the electrophysiological activity of the neurons, individually or collectively per node;

iii) creating a space-time matrix of functional activity of the nodes on the basis of the data, so-called matrix data, obtained in the previous step; and

iv) creating a map representing the cross-temporal and weighted space-time correlation of the activity of each node connected to other nodes in order to provide a set of quantitative data (or matrix data), which prove to be characteristic for each biological model according to the disclosure, in pathological representation configuration. In particular, this consists in generating a “functional activity signature” which will be compared with a “reference library of functional activity signatures” or with a “functional activity signature of the same previously recorded network” or with a “collection of functional activities of the same previously recorded network”, thereby allowing establishing the relative differences between the recorded signatures and the reference signatures.

In the context of the present disclosure, by “functional activity signature”, it should be understood an activity profile, that is to say a possible alteration of the functional activity (i.e. electrical activity) of the neurons in culture in the form of a network in the first compartment of the device according to the disclosure.

In the context of the present disclosure, by “functional activity signature library”, it should be understood a collection of different activity signatures for multiple biological samples (for example 2 or more, advantageously more than 10, preferably more than 100, more than 1,000, or more than 10,000 or even more than 1,000,000 biological samples) which could be identified from one another.

In the context of the disclosure, the comparison with a reference value carried out in step g) consists in comparing at least one characteristic parameter of the state of the neural network of the disclosure consisting of a value obtained from a sample made beforehand (in terms of seconds, minutes, hours, days, months and/or years) with at least one characteristic parameter of the state of the neural network obtained after application of the sample whose effect is to be determined by the method of the disclosure; and/or consisting of a value derived from a reference library or reference collection. In particular,
in the case of the implementation of the in vitro or ex vivo method for determining the effect of a biological sample on a biological model to ensure the monitoring of the state of a subject (i.e. monitor the up or down evolution of a pathology, in particular a neurological and/or nerve condition, the apparition of a pathology, in particular a neurological and/or nerve condition or the effect of a treatment by administration of an agent/test agent), then a monitoring over time of the values of at least one characteristic parameter of the state of the neural network with an evolution criterion (up or down threshold over an analysis period advantageously comprised between 30 sec and 60 min, advantageously between 1 and 50 min, between 2 and 40 min, between 3 and 30 min, and even between 4 and 20 min, preferably between 5 and 10 min, in particular with standard deviation, use of the derivative) is performed;
in the case of a comparison of at least one characteristic parameter of the state of the neural network with an existing functional activity library, all of the recorded parameters i) to vii) should be considered, which allows in particular obtaining a more reliable differential diagnosis, that is to say discriminating between the different possible diagnoses for which there might be identical or similar clinical symptoms because of involvement in these disorders of the same dysfunctional proteins. In this case, the comparison step according to the disclosure comprises, preferably consists of, an absolute comparison with other subjects so-called “true positives” and/or “true negatives” forming groups, or “clusters”, of reference subjects/data; or
the comparison with a reference value carried out in step f) may also consist of an absolute comparison with a library and a monitoring of the state of a subject, as described before.

For example, in the case of determination of the presence or absence of a SARS-COV-2, or COVID-19 infection, step f) of the method of the disclosure comprises, advantageously consists of, an absolute comparison with regards to a functional activity library.

For example, in the case of the establishment of a differential diagnosis of the Alzheimer's disease and the Parkinson's disease, step g) of the method of the disclosure comprises, advantageously consists of, an absolute comparison with regards to a functional activity library. Afterwards, and in order to monitor the evolution of the impairment, a monitoring over time of the values of the connectivity index of the network (parameter iv)) with an evolution criterion (up or down threshold over an average period of time with standard deviation, use of the derivative) is advantageously performed.

For example, in the case of determination of the presence or absence of a head trauma, step g) of the method of the disclosure comprises, advantageously consists in, monitoring the evolution of the impairment by monitoring over time the values of the connectivity index of the network (parameter iv)) with an evolution criterion (up or down over threshold an average period of time with standard deviation, use of the derivative).

For example, the means for converting the functional activity into functional activity data is an algorithmic system for converting electrical and/or electrophysiological data into binary data.

According to the disclosure, it may consist of the conversion of the functional activity signal recorded by the MEA2100-Headstage system (Multichannel systems, Reutlingen, Germany) into a digital signal by an analog-to-digital converter, possibly coupled to an amplifier or set of amplifiers, said converter being directly integrated into the recording equipment. This data stream binary signal is read by the software MEA2100-256-Systems (Multichannel systems, Reutlingen, Germany), provided with the equipment. Alternatively, it may also consist in converting the functional activity signal recorded by the M768tMEA-16 (Axion Biosystems, Atlanta, GA, USA) system into a digital signal by an analog-to-digital converter, possibly coupled to an amplifier or set of amplifiers, said converter being directly integrated into the recording equipment. This binary data stream signal is read by the Axis Navigator (Axion Biosystems, Atlanta, GA, USA) software provided with the equipment.

For example, the means for converting the digital activity of a node into a binary signal of a node is ensured by a thresholding algorithm, conventionally known to a person skilled in the art, which consists in analyzing the background noise of the signal and applying suitable filters in order to apply a binarization threshold over the entire signal.

The method of the disclosure allows applying a biological sample originating from a subject (for example a sample of cerebrospinal fluid, blood, saliva mucus or a test agent) in a bioreceptor comprising, advantageously in the form of, a multi-compartment microfluidic device integrating a relevant cell co-culture (of neuronal and/or non-neuronal and/or explant cells). Preferably, the sample is applied on the neuron culture indirectly, that is to say the application is not done on the neuron culture but on at least one of the associated co-cultures which then acts via the biological interface connected to the culture of neurons by axons and/or synapses extending, for example, in the microchannels of the multi-compartment microfluidic device of the disclosure. Afterwards, the response of the neural network, namely a possible modification of the functional activity of the neural network, following the application of said biological sample is recorded and then analyzed.

In particular, said modification of the functional activity of the neural network may be done by any means known to a person skilled in the art, in particular by:
a modified functional communication, that is to say changes in the efficiency of the cell-to-cell communication wherein the capacity of one or several neuron(s) to activate the target neurons to which they are synaptically or non-synaptically connected, is either increased or reduced or destroyed;
a modification of the networks of axonal and/or dendritic connections, for example by destruction of said axons and/or dendrites;
a modification of one or several cell types impairing the neuronal communication, for example disturbance of the glial cells only within a node, and/or
a modification of the action potentials, that is to say an increase or a decrease in the amplitude, the frequency, the duration, the “threshold” potential enabling a membrane depolarization and/or the rhythm of the action potentials.

These alterations then result in a modification of at least one parameter selected from the group consisting of i) a connection coefficient; ii) an average of the inter-node minimum lengths; iii) an average of the action potentials per second; iv) a network connectivity index; v) a z score; vi) a participation coefficient; and vii) a centrality index of a node. This results in obtaining a functional activity signature, derived from the correlation of said parameters, which is characteristic of a pathological state or not. In fine, the diagnosis is carried out by comparing the functional activity signature thus obtained with a functional activity signature so-called “true positive”, that is to say for which the network markers are true positives.

The result of the implementation of the method of the disclosure in a differential diagnosis allows obtaining a result in less than 72 hours, advantageously in less than 48 h, preferably in less than 24 h, or in less than 12 hours, 6 hours, 3 hours, 2 hours 1 hours, or in less than 30 minutes, 20 minutes, 15 minutes or less than 10 minutes and even immediately by functional analysis of the neural network.

It arises from the foregoing that the innovation of the disclosure therefore lies in the use of network markers as diagnosis markers, in the identification of a relevant neural-cell-explant architecture (i.e. neuronal or non-neuronal or tissue explant cells) for the establishment of a given diagnosis and in the quantification of the network markers by diagnosis type allowing certifying the diagnosis.

One of the advantages of the method of the disclosure is that it allows establishing a reliable differential diagnosis, that is to say a diagnosis allowing differentiating a neurological and/or nerve pathology from another which have close or similar symptoms, or a pathology that has little or no observable symptoms during a primary clinical auscultation.

Another advantage of the method of the disclosure is that it ensures a rapid differential diagnosis.

Furthermore, the method of the disclosure allows improving the specificity/sensitivity ratio. Indeed, the method of the disclosure has a better detection resolution compared to a coupling of conventional molecules (for example ELISA) or to the PCR amplification limits; ensures specificity because of the use of neurons and of at least one associated relevant co-culture allowing modeling a specific cellular and molecular architecture for diagnosing a given pathology.

In addition, the method of the disclosure is inexpensive since it does not require the use of specific machines such as ELISA assays, immunospecific markers, PCRs or other biochemical assay techniques.

Finally, the method of the disclosure is not very constraining and, depending on the modes of sampling intervention, may be barely invasive, since it requires the use of a very small volume of fluid, preferably in the range of microliters, taking account of the implementation of a microfluidic device.

The disclosure also relates to a bioreceptor, as well as a biosensor comprising said bioreceptor, where appropriate, to determine the effect of a biological sample on a biological model. In particular, it consists of a bioreceptor capable of being implemented in the method of the disclosure as described before.

Hence, the disclosure relates to a bioreceptor, as well as a biosensor comprising said bioreceptor where appropriate, for determining the effect of a biological sample on a biological model, comprising, advantageously in the form of, a multi-compartment microfluidic device comprising:

i) at least a first compartment and a second compartment;

ii) at least one means forming a biological interface to enable communication by neural connection between the first and second compartments (FIG. 1);

iii) the culture of at least one type of cells or of an explant per compartment, the first compartment comprising at least the neuron culture in the form of a neural network and the second compartment on which said biological sample could be applied, comprising at least the neuron culture in the form of a neural network and/or the non-neuronal cell culture or an explant culture;

iv) at least one device enabling recording of the functional activity of the neurons over a plurality of measurement points spatially distributed in the first compartment, the device could be combined with a means for converting the functional activity into data.

Preferably, the present disclosure relates to a bioreceptor, as well as a biosensor comprising said bioreceptor where appropriate, for determining the effect of a biological sample on a biological model as described before having the following technical features, considered separately or in combination:
the biological interface comprises, advantageously consists of at least one of the elements selected from the group consisting of fluidic microchannels; PDMS microchannels; a porous membrane, whose porosity is advantageously comprised between 10 nm and 40 μm and whose pore density is advantageously comprised between 10 and 1.10⁹ pores per cm², advantageously between 1.10⁵ and 1.10⁹ pores per cm²; a porous capillary membrane, advantageously it consists of a membrane on which at least one organoid is cultured (i.e. a three-dimensional multicellular structure which reproduces in vitro the microanatomy of an organ) made of polycarbonate, polyester, polyethylene terephthalate and/or polytetrafluoroethylene; a gel; a hydrogel and mixtures thereof;
it further comprises a means for converting the record of the functional activity of the neurons in culture in the first compartment into functional activity data;
it further comprises an analysis means arranged so as to determine at least one characteristic parameter of the state of the neural network in the first compartment from the functional activity data, the at least one parameter being selected from the group consisting of:

i) a connection coefficient;

ii) an average of the inter-node minimum lengths;

iii) an average of the action potentials per second; and

iv) a network connectivity index or “Small World Index”;

v) a z score or “z-score”;

vi) a participation coefficient or “Participation Coefficient”;

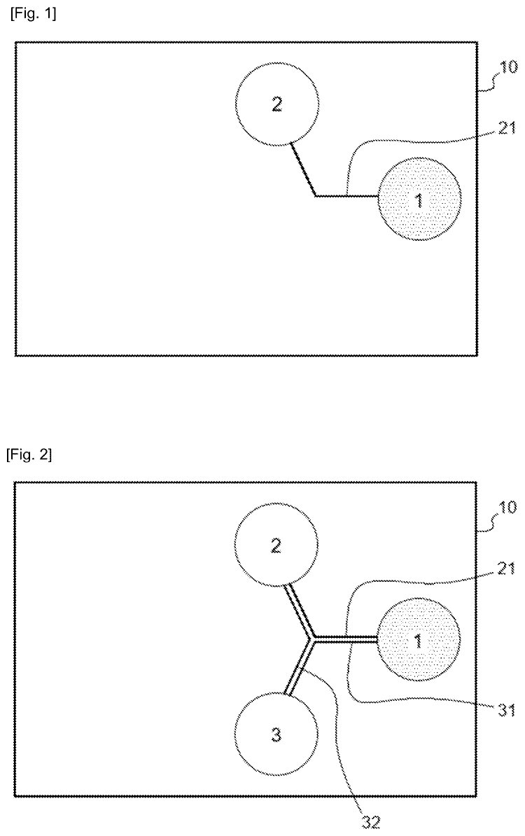

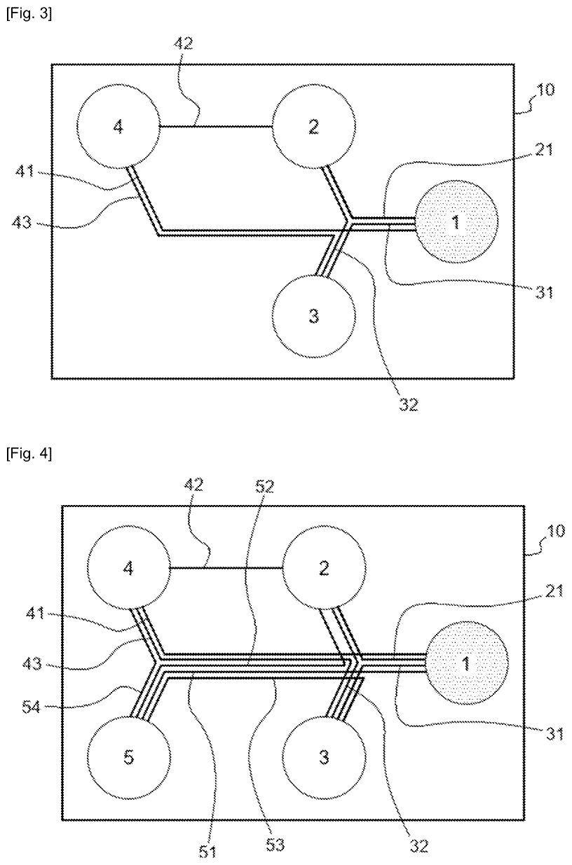

vii) a centrality index of a node;
it further comprises an analysis means arranged so as to determine at least one characteristic parameter of the state of the neural network in the first compartment from the functional activity data, the at least one parameter being selected from the group consisting of: a number of action potential(s), an inter-action potential interval or “ISI”, a coefficient of variation of inter-action potential interval(s), a number of active electrodes, an normalized average rate of action potentials, a number of bursts, a number of electrodes having captured one or several burst(s), an average duration of the bursts, an average of action potentials in a burst, an average of the inter-action potential intervals (ISI) in a burst, an inter-burst interval or “IBI”, a frequency of the bursts, a percentage of the bursts, a network burst number, a network burst frequency, a network burst duration, an average of action potentials in network bursts, an average of the inter-action potential intervals (ISI) in network bursts, a number of electrodes participating in forming network bursts, a percentage of burst in network bursts, a coefficient of variation of inter-burst interval (IBI) in network bursts, a surface area under the cross-correlation curve, and a synchrony index;
it further comprises a means for carrying out a comparison between the at least one characteristic parameter of the state of the neural network and a reference value of at least one characteristic parameter of the state of the neural network in order to determine the effect of a biological sample;
the multi-compartment microfluidic device further comprises a third compartment and at least one means forming a biological interface to enable communication by neural connection between the first and third compartments (1, 3) and/or at least one means forming a biological interface to enable communication by neural connection between the second and third compartments (FIG. 2); advantageously, a fourth compartment and at least one means forming a biological interface to enable communication by neural connection between the first and fourth compartments and/or at least one means forming a biological interface to enable communication by neural connection between the second and fourth compartments and/or at least one means forming a biological interface to enable communication by neural connection between the third and fourth compartments (FIG. 3); preferably, a fifth compartment and at least one means forming a biological interface to enable communication by neural connection between the first and fifth compartments and/or at least one means forming a biological interface to enable communication by neural connection between the second and fifth compartments and/or at least one means forming a biological interface to enable communication by neural connection between the third and fifth compartments and/or at least one means forming a biological interface to enable communication by neural connection between the fourth and fifth compartments (FIG. 4);
it may be included in a portable or non-portable kit, intended to determine the effect of a biological sample on a biological model to, ultimately, establish a differential diagnosis of a neurological and/or nerve condition.

The disclosure also relates to the use of the bioreceptor as described before, as well as a biosensor comprising said bioreceptor, in an in vitro or ex vitro method for diagnosing a neurological or nerve condition, advantageously selected from the group consisting of Alzheimer's disease; Parkinson's disease; head trauma; cerebrovascular accident, thrombotic or embolic occlusion or ischemia; transient ischemic attack; neuronal form of a SARS-COV-2 infection; neuronal intoxication, for example to organophosphorus compounds; analgesia; neuroinflammatory disease, such as multiple sclerosis, otic neuritis, myelitis, lupus, Crohn's disease; hearing impairment by impairment of the hearing nerve; amyotrophic lateral sclerosis; retinal neuropathy, for example diabetes-induced; epilepsy, psoriasis, herpes; meningoencephalitis; isolated lymphocytic meningitis; Guillain-Barre or mononevrite type polyradiculoneuritis; peripheral neuropathy and myelopathy.

The disclosure also relates to the use of the bioreceptor as described before, as well as a biosensor comprising said bioreceptor, in an in vitro or ex vivo method for monitoring a preventive and/or curative treatment of a neurological or nerve condition, advantageously a treatment by gene therapy, cell therapy, axon regeneration therapy, administration of one or several curative and/or preventive and/or anesthetic agent(s).

The disclosure also relates to the use of the bioreceptor as described before, as well as a biosensor comprising said bioreceptor, in a method for rapid and sensitive screening of agents or test agents as potential medicines. Indeed, the device of the disclosure allows identifying the possible therapeutic effect of a test agent as well as assessing the physiologically relevant concentration (for example the amount present in a particular tissue under a prescribed posology) of this agent identified as a medicine. The indirect application of the test agent on a neuron culture in the first compartment produces a recognizable or characteristic signature of functional activity allowing assessing the therapeutic interest of an agent rapidly and efficiently. The present disclosure is illustrated in a non-limiting manner by the following embodiments with reference to the appended figures.

It is considered that, without further specification, in view of the description and of the embodiments, a person skilled in the art should be capable of implementing and using the claimed method and bioreceptor.

The disclosure also relates to an in vitro or ex vivo method for determining the effect of a biological sample on a biological model, comprising the following steps of:

a. Providing a bioreceptor comprising a multi-compartment microfluidic device (10) comprising:

i) at least a first compartment (1) and a second compartment (2);

ii) at least one means forming a biological interface (21) for enabling communication by neural connection between the first and second compartments;

iii) the culture of at least one type of cells or explant per compartment, the first compartment (1) comprising at least the neuron culture in the form of a neural network and the second compartment (2), on which said biological sample may be applied, comprising at least the neuron culture in the form of a neural network and/or the culture of non-neuronal cells or an explant culture;

iv) at least one device (40) enabling recording of the functional activity of the neurons over a plurality of measurement points spatially distributed in the first compartment, the device could be combined with a means (50) for converting the functional activity into data;

b. Directly or indirectly contacting the neurons in culture in the first compartment or the neurons and/or non-neuronal cells or the explant in culture in the second compartment with a biological sample;

c. Carrying out a recording of the functional activity of the neurons in culture in the first compartment (1) over the plurality of measurement points over a measurement duration following contact of the neurons in culture in the first compartment (1) with the biological sample according to step b);

d. Carrying out a conversion of the record of the functional activity of the neurons in culture in the first compartment (1) into functional activity data;

e. Analyzing the functional activity data obtained in step d) and constructing a graph representing the neural network;

f. Determining at least one characteristic parameter of the state of the neural network in the first compartment (1) from the functional activity data, the at least one parameter being selected from the group consisting of:

i) a connection coefficient;

ii) an average of the inter-node minimum lengths;

iii) an average of the action potentials per second;

iv) a network connectivity index or “Small World Index”;

v) a z score or “z-score”;

vi) a participation coefficient or “Participation Coefficient”; and

vii) a centrality index of a node;

g. Carrying out a comparison between the at least one characteristic parameter of the state of the neural network and a reference value of at least one characteristic parameter of the state of the neural network in order to determine the effect of said biological sample.

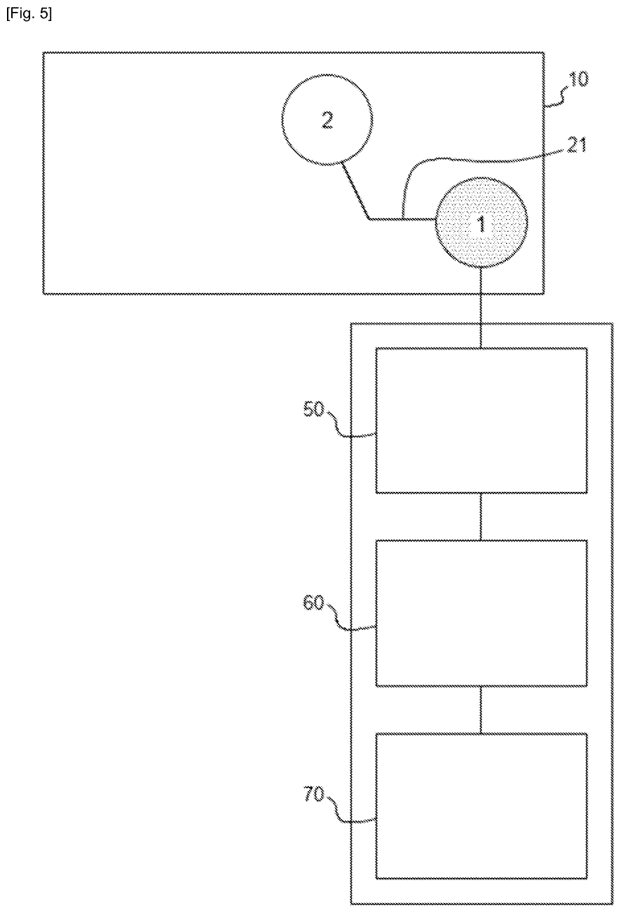

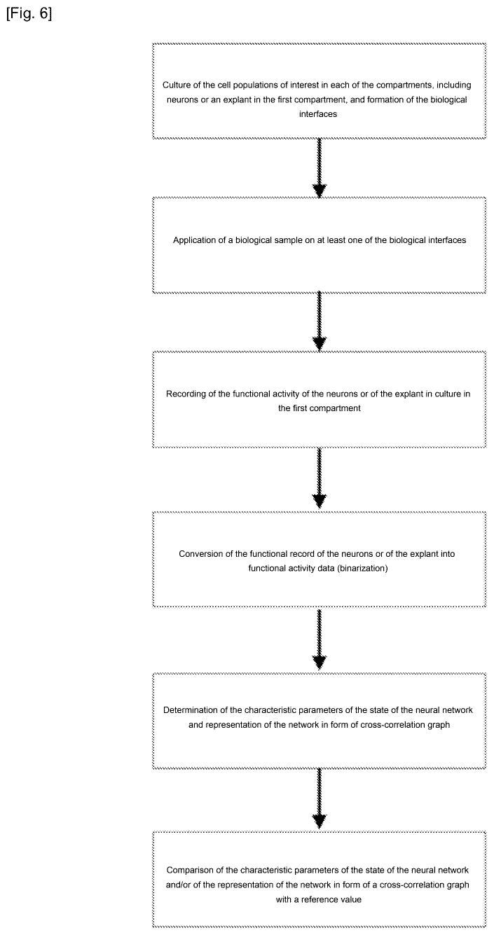

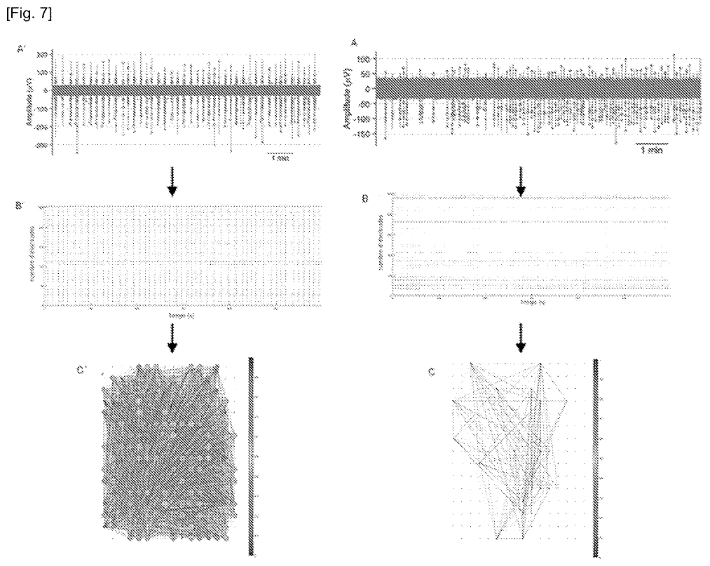

### BRIEF DESCRIPTION OF THE DRAWINGS

[FIG. 1]: Diagram representing the bioreceptor of the disclosure in the form of a microfluidic device comprising 2 compartments.

[FIG. 2]: Diagram representing the bioreceptor of the disclosure in the form of a microfluidic device comprising 3 compartments.

[FIG. 3]: Diagram representing the bioreceptor of the disclosure in the form of a microfluidic device comprising 4 compartments.

[FIG. 4]: Diagram representing the bioreceptor of the disclosure in the form of a microfluidic device comprising 5 compartments.

[FIG. 5]: Diagram representing the different elements necessary for the implementation of the method of the disclosure.

[FIG. 6]: Diagram representing the implemented steps of the method according to the disclosure.

[FIG. 7]: Representation of the implemented steps of the method according to the disclosure and of the data obtained for each of these steps for a healthy subject and a subject suffering from a neurological and/or nerve condition.

### DETAILED DESCRIPTION OF THE DRAWINGS

As already mentioned before, the disclosure relates, in a first embodiment as represented by FIGS. 1 and 5, to a bioreceptor comprising a multi-compartment microfluidic device comprising, in a first embodiment, a first compartment 1 and a second compartment 2, each comprising the culture of at least one type of cells or explant; and at least one means forming a biological interface 21 to enable communication by neuronal connection between the first and second compartments 1, 2.

The bioreceptor of the disclosure also comprises at least one device 40 allowing recording the functional activity of the neurons over a plurality of measurement points spatially distributed in the first compartment, the device could be combined with a means 50 for converting the functional activity into functional activity data, a means 60 for analyzing these functional activity data arranged so as to determine at least one characteristic parameter of the state of the neural network in the first compartment from the functional activity data, and a means 70 for carrying out a comparison between the at least one characteristic parameter of the state of the neural network and a reference value of at least one characteristic parameter of the state of the neural network in order to determine the effect of a biological sample.

Said at least one parameter may be selected from the group consisting of:

i) a connection coefficient;

ii) an average of the inter-node minimum lengths;

iii) an average of the action potentials per second; and

iv) a network connectivity index or “Small World Index”;

v) a z score or “z-score”;

vi) a participation coefficient or “Participation Coefficient”;

vii) a centrality index of a node.

Said at least one parameter may also be selected from the group consisting of: a number of action potential(s), an inter-action potential interval or “ISI”, a coefficient of variation of inter-action potential interval(s), a number of active electrodes, an average rate of normalized action potentials, a number of bursts, a number of electrode(s) having captured one or several burst(s), an average duration of the bursts, an average of action potentials in a burst, an average of the inter-action potential intervals (ISI) in a burst, an inter-burst interval or “IBI”, a frequency of the bursts, a percentage of the bursts, a number of network bursts, a frequency of network bursts, a network burst duration, an average of action potentials in network bursts, an average of the inter-action potential intervals (ISI) in network bursts, a number of electrodes participating in forming network bursts, a percentage of bursts in network bursts, a coefficient of variation of inter-burst intervals (IBI) in network bursts, a surface area under the cross-correlation curve, and a synchrony index.

When two parameters or more are used to characterize the state of the neural network in the first compartment from the functional activity data, this may consist of a combination of parameters belonging to the same group or a combination of parameters each derived from one of the aforementioned two groups.

In a second embodiment as represented by FIG. 2, the bioreceptor of the disclosure further comprises a third compartment 3; at least one means 31 forming a biological interface to enable communication by neuronal connection between the first and third compartments 1, 3 and/or at least one means 32 forming a biological interface to enable communication by neuronal connection between the second and third compartments 2, 3.

In a third embodiment as represented by FIG. 3, the bioreceptor of the disclosure further comprises a fourth compartment 4; at least one means 41 forming a biological interface to enable communication by neuronal connection between the first and fourth compartments 1, 4 and/or at least one means 42 forming a biological interface to enable communication by neuronal connection between the second and fourth compartments 2, 4 and/or at least one means 43 forming a biological interface to enable communication by neuronal connection between the third and fourth compartments 3, 4.

In a fourth embodiment as represented by FIG. 4, the bioreceptor of the disclosure further comprises a fifth compartment 5; at least one means 51 forming a biological interface to enable communication by neuronal connection between the first and fifth compartments 1, 5 and/or at least one means 52 forming a biological interface to enable communication by neuronal connection between the second and fifth compartments 2, 5 and/or at least one means 53 forming a biological interface to enable communication by neuronal connection between the third and fifth compartments 3, 5 and/or at least one means 54 forming a biological interface to enable communication by neuronal connection between the fourth and fifth compartments 4, 5.

As represented by FIGS. 6 and 7 (including the sub-FIGS. 7A, 7B′, 7C′, 7A, 7B and 7C), the implementation of the in vitro or ex vivo method for determining the effect of a biological sample on a biological model according to the disclosure allows obtaining a record of the functional activity of the neurons (FIGS. 7A′ and 7A) in culture in the first compartment 1, that is to say, a record of the extracellular activity of the neurons which are reflected by peaks so-called “spikes” which, when a neural network is synchronized (or in non-pathological condition), appear periodically. Thus, a strong activity of the neural network in culture in the first compartment 1 is observed by the presence of burst of peaks so-called “burst”. On the contrary, other types of neurons, like GABAergic neurons, do not present a synchronized functional activity (data not shown). In this case, the possible disturbance of the functional activity will not result in a desynchronization of the “spikes” and/or a decrease or absence of the “bursts”, but not a modification of the characteristic parameters of the state of the neural network according to the disclosure.

This recording of the functional activity of the neurons in the form of a network in culture in the first compartment is subjected to a conversion means 50 allowing obtaining functional activity data, in particular binary data (FIGS. 7B′ and 7B).

In turn, these functional activity data are submitted to an analysis means 60 arranged so as to determine at least one characteristic parameter of the state of the neural network in the first compartment from the functional activity data, the at least one parameter being selected from the group consisting of:

i) a connection coefficient;

ii) an average of the inter-node minimum lengths;

iii) an average of the action potentials per second; and

iv) a network connectivity index or “Small World Index”;

v) a z score or “z-score”;

vi) a participation coefficient or “Participation Coefficient”;

vii) a centrality index of a node.

In fine, these characteristic data/parameters of the state of the neural network are correlated and may be represented in the form of a cross-correlation graph (FIGS. 7C′ and 7C).

The bioreceptor comprises a means 70 for carrying out a comparison between the at least one characteristic parameter of the state of the neural network and a reference value of at least one characteristic parameter of the state of the neural network in order to determine the effect of the application of a biological sample on a biological model and, consequently, determine the presence or absence of a neurological and/or nerve condition in a subject, or to identify new therapeutic solutions (new molecules or new posology) from screening of test agents.

In particular, when the average of the action potentials per second has a value higher than 0.5, then it is possible to analyze at least one parameter being selected from the group consisting of:

i) a connection coefficient;

ii) an average of the inter-node minimum lengths; and

iv) a network connectivity index or “Small World Index”;

v) a z score or “z-score”;

vi) a participation coefficient or “Participation Coefficient”;

vii) a centrality index of a node;

In particular, the analysis comprises, advantageously consists of, the determination of the value of the average connection coefficient ratio of the inter-node minimum lengths, allowing, in fine, determining the connectivity index of the network which will then be the parameter implemented in the comparison step.

### EMBODIMENTS

The present disclosure will be illustrated further in connection with a bioreceptor comprising a multi-compartment microfluidic device 10 comprising 2 compartments (Examples 1 to 3) or 5 compartments (Example 4). Nonetheless, these examples in no way limit the disclosure.

### Example 1: Diagnosis of Alzheimer's Disease

### 1. Context

More than 10 million people in Europe suffer from neurodegenerative diseases such as Alzheimer's disease (AD) and this number tends to double in less than 20 years. There is no curative treatment for these disorders and the search for new treatments is made difficult by the complexity of establishing a reliable differential diagnosis. The first lesions of the AD appear in the hippocampus, which is an area of the brain involved in the memorization processes (recording, restitution and organization of the memories) and in the management of the emotions, before progressively diffusing in the direction of the outer areas following the connections established between the different brain regions. The hippocampus is a structure composed of glial cells (astrocytes or microglial cells) and, neurons the majority of which are glutamatergic and GABAergic neurons.

### 2. Equipment and Methods

The bioreceptor of the disclosure is in the form of a multi-compartment device wherein a first compartment comprises the culture of human glutamatergic neurons derived from stem cells and, a second compartment comprises the culture of human GABAergic neurons derived from stem cells.

Gluamatergic neurons are stored in nitrogen at a temperature of about −200° C. A progressive thawing step is performed according to conventional techniques widely known to a person skilled in the art. Afterwards, the thawed cells are set in about 10 mL of a dedicated culture medium at 37° C. The aliquot is centrifuged and then treated using an automatic counter or with a Malassez cell in order to determine the concentration of glutamatergic neurons and the dilutions to be performed. Afterwards, the aliquot is diluted and seeded in the first compartment of a device according to the disclosure processed beforehand according to conventional techniques widely known to a person skilled in the art to facilitate the adhesion of glutamatergic neurons to the substrate.

GABAergic neurons are cultured in the second compartment of said device, according to the same protocol as that one detailed hereinabove.

The functional activity of the glutamatergic neurons thus cultured is recorded by means of arrays of planar microelectrodes (MEA) 256MEA100/30iR-ITO-w/o (Multichannel systems, Reutlingen, Germany) consisting of 30 μm diameter electrodes spaced apart by 100 μm of electrodes. This recording is performed for 10 min thanks to the MEA2100-256-Systems software (Multichannel systems, Reutlingen, Germany). The technology of the microelectrode arrays allows recording the functional and spontaneous extracellular activity of the neurons as a network connectivity marker.

For the functional record, two conditions have been tested:
“test” condition: sample of cerebrospinal fluid of a subject likely to suffer from MA; and
“reference” condition: sample of cerebrospinal fluid of a so-called “true negative” subject, serving as a reference value.

In particular, as regards the “reference” condition, it should be noted that it consists of a sample obtained from a healthy subject and presenting a negative result to the conventional AD test. In other words, it consists of a sample reflecting a so-called “true negative” diagnosis.

### 3. Results

The results are represented by FIGS. 7A′, 7B′, 7C′, 7A, 7B and 7C.

During the electrophysiological recording, glutamatergic neurons in culture have shown a functional activity represented by points (FIGS. 7A′ and 7A). These peaks are detected in time and space by the software algorithm MEA2100-256-Systems which then assigns to a value, identified by each of the points on the peaks.

On the basis of said values identified by the algorithm, a matrix (raster plot or rasterplot) is generated in order to visualize the activity of each active electrode as a function of time (FIGS. 7B′ and 7B). This graph allows assessing the synchronicity of a neural network, namely the ability of the neurons to have an activity at the same time. It clearly arises from these graphs that the glutamatergic neural network is synchronized under “reference” conditions (FIG. 7B′) and desynchronized under “test” conditions (FIG. 7B).

Furthermore, the optimum structure of the network is defined by quantitative parameters obtained by a cross-correlation algorithm and allows estimating the quality of the network connectivity. In this case, the average of the action potentials per second has a value higher than 0.5; which enables the determination of the value of the average connection coefficient ratio of the inter-node minimum lengths, and, in fine, the determination of the value of the connectivity index of the network.

The processing and analysis, as described before, of the obtained data allows representing a neural network, defined by nodes and interactions with one another (FIG. 7C′ and 7C). The cross-correlation is an algorithm representing the degree of the neural network and allows estimating the state function of the network.

The analysis of the network shows that the neural network under the “test” condition (FIG. 7C) has less connections than the neural network under the “reference” condition (FIG. 7C′).

### 4. Conclusion

It arises from the implementation of the method of the disclosure that the sample of the “test” condition is derived from a subject suffering from a neurological and/or nerve condition.

The comparison of the representation of the neural network under the “test” conditions (FIG. 7C) with a library of functional activity signatures allows establishing a rapid and reliable diagnosis of Alzheimer's disease.

Afterwards, a monitoring of the state of a subject (i.e. monitoring the up or down evolution of the AD) is performed by monitoring over time the values of the connectivity index of the network (parameter iv)) with an evolution criterion (up or down threshold over an average period of time with standard deviation, use of the derivative) with respect to the reference value corresponding to the value obtained at the time of the diagnosis of the disease, that is to say the value obtained as described before).

### Example 2: Diagnosis of a Head Trauma that Occured During a Rugby Match

### 1. Context

In some risky sports, like rugby, players are very exposed to the risk of head trauma (or concussion). It may be defined as a short-duration dysfunction of the brain functions in the absence of macro-or microscopic lesions. The frequency of the commotions is increasing these last 15 years. The incidence in the rugby is observed between 4.1 and 7.9/1,000 hour-player in the course of match. Currently, the diagnosis is based on the establishment of a Glasgow score established by clinical observations and symptoms. The concussion may result from a hit other than on the head, the player may have clinical signs and symptoms that are very low or even unnoticed, without loss of knowledge, which may make the diagnosis more difficult and slower. The time between the diagnosis and the prognosis of the player might be very long and immobilize the player for several days, or weeks and sometimes have harmful, heavy and long-term consequences.

### 2. Equipment and Methods

The bioreceptor of the disclosure is in the form of a multi-compartment device wherein a first compartment comprises the culture of human sensory neurons derived from stem cells and, a second compartment comprises the culture of cells of the oral mucosa. Cf. the protocol detailed in point 2. of Example 1.

For the functional record, two conditions have been tested:
“test” condition: saliva sample obtained from a subject who has experienced a shock during a rugby match; and
“reference” condition: saliva sample obtained from a healthy subject and presenting a negative result to the conventional test for detecting a head trauma. In other words, it consists of a sample reporting a so-called “true negative” diagnosis.

### 3. Results

Data are obtained according to the protocol detailed in point 3. of Example 1.

### 4. Conclusion

It arises from the implementation of the method of the disclosure that the sample of the “test” condition is derived from a subject suffering from a neurological and/or nerve condition.

The comparison of the representation of the neural network under “test” conditions with a library of functional activity signatures allows establishing a rapid and reliable diagnosis of the presence of a head trauma allowing ensuring a fast and efficient management of the subject.

### Example 3: Diagnosis of COVID-19 Disease

### 1. Context

Coronaviruses are known to lead to severe acute respiratory syndromes. These have been at the origin of three mortal epidemics during the 21th century. SARS-COV-2, responsible for the COVID-19 disease, is at the origin of the most recent pandemic with a significantly high mortality rate and dramatic economic costs. In Europe, the cumulative mortality is 34% and is variable from one European country to another, correlated with the emergence of new variants of SARS-COV-2and a strong heterogeneity of the symptoms (asymptomatic persons, benign forms, death of the individual). The ability of coronaviruses to invade the central nervous system had already been described during the two preceding epidemies caused by SARS-COV-1 and MERS-COV. The described neurological disorders have different levels of seriousness ranging from simple headaches, temporary confusions to cerebral vascular accidents and convulsions in the most severe forms. As many other airborne viral diseases, the infection of the upper respiratory tracts is the first entry gate in the body. All the more so since olfactory attacks, including the loss of odor detection (anosmic), following a SARS-COV-2 infection, remain one of the most common and predictive symptoms of infection even with current tests. In particular, it has been stated that neuroinvasion takes place via the nasal route. Indeed, the presence of intact viral particles in the support cells of the olfactory mucosa has been demonstrated, underlying that this place could be the seat of viral replication explaining by the way the loss of taste and smell. The assumption regarding neuroinvasion is that viral propagation is done via the olfactory bulb (the seat of processing of the olfactory sensory information) before entry into the central nervous system via the cranial nerves.

### 2. Equipment and Methods

The bioreceptor of the disclosure is in the form of a multi-compartment device wherein a first compartment comprises the culture of human mitral cells derived from the olfactory bulb and, a second compartment comprises the culture of cells of the nasal mucosa. Cf. the protocol detailed in point 2. of Example 1.

As regards the functional record, two conditions have been tested:
“test” condition: nasopharyngeal sample obtained from a subject; and
“reference” condition: nasopharyngeal sample obtained from a healthy subject and presenting a negative result to the conventional test for detecting the COVID-19 disease. In other words, it consists of a sample reflecting a so-called “true negative” diagnosis.

### 3. Results

Data are obtained according to the protocol detailed in point 3. of Example 1.

### 4. Conclusion

It arises from the implementation of the method of the disclosure that the sample of the “test” condition is derived from a subject suffering from a neurological and/or nerve condition.

The comparison of the representation of the neural network under “test” conditions with a library of functional activity signatures allows establishing a rapid and reliable diagnosis of attainment by the COVID-19 disease.

### Example 4: Diagnosis of Parkinson's Disease

### 1. Context

Parkinson's disease (PD) is the second most widespread neurodegenerative disease, after Alzheimer's disease, with a prevalence of 4% of persons aged more than 80 years. From a clinical point of view, it has motor symptoms, affecting the walk and the movements of the body, associated with non-motor symptoms. Dementia is a current symptom in Parkinson's disease and corresponds either to parkinsonian dementia or to dementia with Lewy bodies.

From a neuropathological point of view, the PD is characterized by a loss of dopaminergic neurons in the black substance (SN, an anatomical region belonging to the ganglia of the base) and by the presence of intracellular inclusions so-called Lewy bodies. It is in these Lewy bodies that the a-synuclein protein is located, which, when it is poorly folded and shaped, leads to a neurotoxicity cascade.

The axonal projections of the SN extend towards the putamen and the caudate nucleus (which form the striatum) where there is a connection series to the pallidus globus and the subthalamic nucleus. In the PD, the degeneration of the nigrostriatal route (route between the black substance and the striatum) is the first cause of the motor symptoms. A key consequence of the death of dopaminergic neurons in the SN and of the reduction of dopamine is the disturbance of the dopaminergic signaling of the ganglia of the base to the rest of the brain. The motor circuit of the ganglia of the base controls the movement. In this circuit, a direct route and an indirect route are found. The direct route is composed of 5 anatomical regions: The cortex, the striatum, the pars compacta black substance, the thalamus and the association of the inner pallidus globus and the reticularis black substance.

### 2. Equipment and Methods

The bioreceptor of the disclosure is in the form of a multi-compartment device wherein a first compartment comprises the culture of human glutamatergic and GABAergic cells derived from pluripotent stem cells, a second compartment comprises the culture of GABAergic neurons, a third compartment comprises the culture of glutamatergic neurons, a fourth compartment comprises the culture of GABAergic neurons and a fifth compartment comprising dopaminergic neurons. These five compartments correspond to the five anatomical regions of the lymph node loop of the base. Cf. the protocol detailed in point 2. of Example 1.

For the functional record, two conditions have been tested:
“test” condition: sample of cerebrospinal fluid of a subject likely to suffer from the Parkinson's disease; and
“reference” condition: sample of cerebrospinal fluid of a so-called “true negative” subject, serving as a reference value.

In particular, as regards the “reference” condition, it should be noted that it consists of a sample obtained from a healthy subject and presenting a negative result to the conventional Parkinson's disease test. In other words, it consists of a sample reflecting a so-called “true negative” diagnosis.

### 3. Results

Data are obtained according to the protocol detailed in point 3. of Example 1.

### 4. Conclusion

It arises from the implementation of the method of the disclosure that the sample of the “test” condition is derived from a subject suffering from a neurological and/or nerve condition.

The comparison of the representation of the neural network under “test” conditions with a library of functional activity signatures allows establishing a rapid and reliable diagnosis of attainment by Parkinson's disease.

1. An in vitro or ex vitro method for determining the effect of a biological sample on a biological model, the method including the following steps of:
a. Providing a bioreceptor comprising a multi-compartment microfluidic device comprising:
i) at least a first compartment and a second compartment;
ii) at least one means forming a biological interface for enabling communication by neural connection between the first and second compartments;
iii) the first compartment comprising at least the neuron culture in the form of a neural network and the second compartment comprising at least the neuron culture in the form of a neural network and/or the culture of non-neuronal cells or an explant culture;
iv) at least one device enabling recording of the functional activity of the neurons over a plurality of measurement points spatially distributed in the first compartment;
b. Directly or indirectly contacting the neurons and/or non-neuronal cells or the explant in culture in the second compartment with said biological sample;
c. Carrying out a recording of the functional activity of the neurons in culture in the first compartment over the plurality of measurement points over a measurement duration following contacting according to step b);
d. Carrying out a conversion of the record of the functional activity of the neurons in culture in the first compartment into functional activity data;
e. Analyzing the functional activity data obtained in step d);
f. Determining at least one characteristic parameter of the state of the neural network in the first compartment from the functional activity data;
g. Carrying out a comparison between the at least one characteristic parameter of the state of the neural network and a reference value of at least one characteristic parameter of the state of the neural network in order to determine the effect of said biological sample.

2. The method according to claim 1, wherein the at least one characteristic parameter of the state of the neural network in the first compartment is selected from the group consisting of: a number of action potential(s), an action inter-potential interval or “ISI”, a variation coefficient of inter-action potential interval(s), a number of active electrodes, an average rate of normalized action potentials, a number of bursts, a number of electrode(s) having captured one or several burst(s), an average duration of the bursts, an average of action potentials in a burst, an average of the inter-action potential intervals (ISI) in a burst, an inter-burst interval or “IBI”, a frequency of the bursts, a percentage of the bursts, a number of network bursts, a network burst frequency, a network burst duration, an average of action potentials in network bursts, an average of the inter-action potential intervals (ISI) in network bursts, a number of electrodes participating in forming network bursts, a burst percentage in network bursts, an inter-burst interval variation coefficient (IBI) in network bursts, a surface area under the cross-correlation curve, and a synchrony index.

3. The method according to claim 1, wherein the at least one characteristic parameter of the state of the neural network in the first compartment is selected from the group consisting of:
i) a connection coefficient;
ii) an average of the inter-node minimum lengths;
iii) an average of the action potentials per second;
iv) a network connectivity index or “Small World Index”;
v) a z score or “z-score”;
vi) a participation coefficient or “Participation Coefficient”; and
vii) a centrality index of a node.

4. The method according to claim 3, wherein step f) comprises determining two parameters of the state of the neural network in the first compartment from the functional activity data, said parameters being selected from the group consisting of:
i) a connection coefficient;
ii) an average of the inter-node minimum lengths;
iii) an average of the action potentials per second;
iv) a network connectivity index or “Small World Index”;
v) a z score or “z-score”;
vi) a participation coefficient or “Participation Coefficient”; and
vii) a centrality index of a node.

5. The method according to claim 3, wherein step g) comprises a step of comparing with thresholds, namely:
the average of the action potentials per second is compared with an average threshold of the action potentials per second whose value is greater than or equal, advantageously strictly greater than 0.5; and/or
the connection coefficient is compared with a connection coefficient threshold whose value is greater than or equal to 0, advantageously comprised between 0 and 1; and/or
the average of the inter-node minimum lengths is compared to an average threshold of the inter-node minimum lengths whose value is greater than or equal to 1, advantageously 1.5; and/or
the connectivity index of the inter-node connectivity network is compared with an inter-node connectivity threshold whose value is greater than or equal to 0, advantageously comprised between 0 and 1; and/or
the score z or “z-score” has a value greater than or equal to 0; and/or
the participation coefficient or “Participation Coefficient” has a value greater than or equal to 0; and/or
the centrality index of a node has a value greater than or equal to 0.

6. The method according to claim 1, wherein step g) comprises a comparison of at least one characteristic parameter of the state of the neural network as defined by step f) with a reference library of functional activity signatures.

7. The method according to claim 3, wherein step g) comprises monitoring the values of the parameter iv) with an up or down evolution criterion over an analysis period.

8. The method according to claim 3, wherein in step f) the determination of at least one characteristic parameter of the state of the neural network being selected from the group consisting of:
i) a connection coefficient;
ii) an average of the inter-node minimum lengths;
iii) a network connectivity index or “Small World Index”;
iv) a z score or “z-score”;
v) a participation coefficient or “Participation Coefficient”; and
vi) a centrality index of a node;
is performed when the average of the action potentials per second is higher than 0.5.

9. The method according to claim 8, wherein the determination of at least one characteristic parameter of the state of the neural network comprises, advantageously comprises determining the connectivity index of the network which comprises the value of the ratio of the connection coefficient of the inter-node minimum lengths.

10. The method according to claim 1, wherein the device allowing recording the functional activity of the neurons over a plurality of measurement points spatially distributed in the first compartment according to step a.iv) is a device enabling an indirect contact recording with the cultured cells, selected from the group consisting of:
a device for recording activity by arrays of planar or non-planar microelectrode, semi-solid electrodes, by amperometry or voltammetry;
a fluorescence imaging recording device, such as calcium imaging or transmembrane ion flow imaging; and
a device for recording intracellular, extracellular, or patch-clamp electrophysiological activity in whole cell, attached cell, inside-out or outside-out configuration.

11. The method according to claim 1, wherein step d) of carrying out a conversion of the record of the functional activity of the neurons in culture in the first compartment into functional activity data is carried out via a means for converting the functional activity of neurons into functional activity data, the conversion means being an algorithmic system for converting electrical and/or electrophysiological data into binary data.

12. The method according to claim 1, wherein the duration of measurement of the record of the functional activity of the neurons in culture in the first compartment according to step c) is comprised between 300 ms and 20 min.

13. The method according to claim 1, wherein the multi-compartment microfluidic device further comprises:
i) a third compartment comprising at least the neuron culture in the form of a neural network and/or the non-neuronal cell culture; and
ii) at least one means forming a biological interface to enable communication by neural connection between the first and third compartments and/or at least one means forming a biological interface to enable communication by neural connection between the second and third compartments.

14. The method according to claim 13, wherein the multi-compartment microfluidic device further comprises:
iii) a fourth compartment comprising at least the neuron culture in the form of a neural network and/or the non-neuronal cell culture; and
iv) at least one means forming a biological interface to enable communication by neural connection between the first and fourth compartments and/or at least one means forming a biological interface to enable communication by neural connection between the second and fourth compartments and/or at least one means forming a biological interface to enable communication by neural connection between the third and fourth compartments.

15. The method according to claim 14, wherein the multi-compartment microfluidic device further comprises:
v) a fifth compartment comprising at least the neuron culture in the form of a neural network and/or the non-neuronal cell culture; and
vi) at least one means forming a biological interface to enable communication by neural connection between the first and fifth compartments and/or at least one means forming a biological interface to enable communication by neural connection between the second and fifth compartments and/or at least one means forming a biological interface to enable communication by neural connection between the third and fifth compartments and/or at least one means forming a biological interface to enable communication by neural connection between the fourth and fifth compartments.

16. The method according to claim 1, wherein the contacting according to step b) is indirect in that the biological sample is applied on the biological interface of the second compartment and/or on at least one of the biological interfaces of the third compartment and/or on at least one of the biological interfaces of the fourth compartment and/or on at least one of the biological interfaces of the fifth compartment.

17. The method according to claim 1, wherein:
the neurons are selected from the group consisting of glutamatergic, GABAergic, serotoninergic, cholinergic, dopaminergic, adrenergic, noradrenergic, sensory neurons and motor neurons; and/or
the non-neuronal cells are selected from the group consisting of glial, epithelial, conjunctive, thyroid, fat, blood, immune, bone, cartilage, gastric, pancreatic, hepatic, intestinal, pulmonary, endothelial, muscle, vascular, cardiac, mesenchymal cells, retinal pigment epithelium cells, retinal pigment epithelium cells and retinal cells; and/or
the explant is tissue of cerebral, epithelial, ocular, thyroid, fat, vascular, bone, cartilage, gastric, pancreatic, hepatic, intestinal, pulmonary, endothelial, muscle, retinal, cardiac and placental origin.

18. The method according to claim 1, wherein the biological sample is selected from the group consisting of blood, saliva, urine, tears, sweat, sputum, mucus, pus, lymph, cerebrospinal fluid, nasopharyngeal secretions, oropharyngeal secretions, synovia, pleural fluid, peritoneal fluid, pericardial fluid, aqueous humor, amniotic fluid and plasma.

19. The method according to claim 1, wherein the biological sample is an agent or a test agent.

20. A bioreceptor for determining the effect of a biological sample on a biological model, comprising a multi-compartment microfluidic device comprising:
i) at least a first compartment and a second compartment;
ii) at least one means forming a biological interface to enable communication by neural connection between the first and second compartments;
iii) the first compartment comprising at least the neuron culture in the form of a neural network and the second compartment on which said biological sample could be applied, comprising at least the neuron culture in the form of a neural network and/or the non-neuronal cell culture or an explant culture;
iv) at least one device enabling recording of the functional activity of the neurons over a plurality of measurement points spatially distributed in the first compartment.

21. The biosensor comprising a bioreceptor according to claim 20, wherein the biosensor further comprises a means for converting the record of the functional activity of the neurons in culture in the first compartment into functional activity data.

22. The biosensor according to claim 21, wherein the biosensor further comprises an analysis means arranged so as to determine at least one characteristic parameter of the state of the neural network in the first compartment from the functional activity data, the at least one parameter being selected from the group consisting of:
i) a connection coefficient;
ii) an average of the inter-node minimum lengths;
iii) an average of the action potentials per second;
iv) a network connectivity index or “Small World Index”;
v) a z score or “z-score”;
vi) a participation coefficient or “Participation Coefficient”; and
vii) a centrality index of a node.

23. The biosensor according to claim 21, wherein the biosensor further comprises an analysis means arranged to determine at least one characteristic parameter of the state of the neural network in the first compartment from the functional activity data, the at least one parameter being selected from the group consisting of: a number of action potential(s), an inter-action potential interval, or “ISI”, a variation coefficient of inter-action potential interval(s), a number of active electrodes, an average rate of normalized action potential, a number of bursts, a number of electrode(s) having captured up one or several burst(s), an average duration of the bursts, an average of action potentials in a burst, an average of the inter-action potential intervals (ISI) in a burst, an inter-burst interval or “IBI”, a frequency of the bursts, a percentage of the bursts, a number of network bursts, a network burst frequency, a network burst duration, an average of action potentials in network bursts, an average of inter-action potential intervals (ISI) in network bursts, a number of electrodes participating in forming network bursts, a burst percentage in network bursts, an inter-burst interval variation coefficient (IBI) in network bursts, a surface area under the cross-correlation curve, and a synchrony index.

24. The biosensor according to claim 22, wherein the biosensor further comprises a means for carrying out a comparison between the at least one characteristic parameter of the state of the neural network and a reference value of said at least one characteristic parameter of the state of the neural network in order to determine the effect of said biological sample.

25. A use of the bioreceptor according to claim 20 in an in vitro or ex vitro method for diagnosing a neurological and/or nerve condition, advantageously selected from the group consisting of Alzheimer's disease; Parkinson's disease; head trauma;
cerebrovascular accident, thrombotic or embolic occlusion or ischemia; transient ischemic attack; neuronal form of a SARS-COV-2 infection (=covid-19); neuronal intoxication, for example to organophosphorus compounds; analgesia;
neuroinflammatory disease, such as multiple sclerosis, otic neuritis, myelitis, lupus, Crohn's disease; hearing impairment by impairment of the hearing nerve; amyotrophic lateral sclerosis; retinal neuropathy, for example diabetes-induced; epilepsy, psoriasis, herpes; meningoencephalitis; isolated lymphocytic meningitis; Guillain-Barre or mononevrite type polyradiculoneuritis; peripheral neuropathy and myelopathy.

26. A use of the bioreceptor according to claim 20 in an in vitro or ex vitro method for monitoring a preventive and/or curative treatment of a neurological or nerve condition, advantageously a treatment by gene therapy, cell therapy, axon regeneration therapy, administration of one or several curative and/or preventive and/or anesthetic agent(s).

27. A use of the bioreceptor according to claim 20 in an in vitro or ex vitro method for identifying and/or characterizing the therapeutic properties of an agent and/or a threshold concentration of an agent.
